(19) United States
(12) Patent Application Publication
(10) Pub. No.: US 2023/0333114 A1
(43) Pub. Date: Oct. 19, 2023

(54) SYSTEMS AND METHODS FOR THE AMPLIFIED DETECTION OF MOLECULES ON MICROPARTICLES

(71) Applicant: THE REGENTS OF THE UNIVERSITY OF CALIFORNIA, Oakland, CA (US)

(72) Inventors: Dino Di Carlo, Los Angeles, CA (US); Yilian Wang, Los Angeles, CA (US); Vishwesh Nilesh Shah, Los Angeles, CA (US)

(73) Assignee: THE REGENTS OF THE UNIVERSITY OF CALIFORNIA, Oakland, CA (US)

(21) Appl. No.: 18/044,371

(22) PCT Filed: Sep. 8, 2021
(86) PCT No.: PCT/US2021/049493

§ 371 Date: Mar. 7, 2023

Related U.S. Application Data
(60) Provisional application No. 63/076,259, filed on Sep. 9, 2020.

(51) Int. Cl. G01N 33/58; G01N 33/543; G01N 15/14

(52) U.S. Cl. CPC G01N 33/581; G01N 33/582; G01N 33/54313; G01N 15/1459; G01N 2333/908; G01N 2015/149

(57) ABSTRACT

A particle-based assay system is disclosed that uses hydrogel microparticles that capture analytes of interest from a sample which are subsequently bound with catalytic reporter complexes. Catalytic reporter complexes bound to the hydrogel microparticles generate signals that are accumulated in the vicinity of the hydrogel microparticle at high concentration (or on or within the hydrogel microparticles). In some circumstances, the reporter complex-bound hydrogel microparticles are encapsulated in an emulsion. Preferably, the emulsion is substantially uniform and contains one hydrogel microparticle per droplet. The accumulated signal generated by the catalytic reporter complexes is contained inside the emulsion, and/or optionally immobilized onto or inside the hydrogel microparticle. Signals are read and analyzed using optical instruments such as flow cytometers. Breaking the emulsion prior to signal analysis is optional. In some embodiments, a sample is introduced to hydrogel microparticles in a dried state to concentrate analytes of interest.

### RELATED APPLICATION

This Application claims priority to U.S. Provisional Patent Application No. 63/076,259 filed on Sep. 9, 2020, which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number 1648451, awarded by the National Science Foundation. The government has certain rights in the invention.

### TECHNICAL FIELD

The technical field relates to hydrogel microparticles that are used to capture analytes of interest from a sample. In particular, the hydrogel microparticles bind to the capture analytes and then are subsequently bound with catalytic reporter complexes that generate signals that accumulate on or within the hydrogel microparticles or are sequestered in close proximity to the hydrogel microparticle in an emulsion.

### BACKGROUND

The detection, measurement, and analysis of protein biomarkers are important in clinical diagnostics and life sciences research. Enzyme-linked immunosorbent assay (ELISA) is commonly used to detect and quantify protein biomarkers in a complex mixture. The method selectively immobilizes the analytes of interest onto a chemically modified surface, usually the surface of wells in well plates by specific antigen-antibody binding, and determines the quantity of the analytes of interest by measuring signals generated by an enzymatic reporter turning substrate molecules into signaling molecules. Leveraging the high specificity of antigen-antibody interaction, and amplified signals from the enzymatic reporter, ELISA was among the most specific and sensitive protein detection methods and has become a standard and fundamental biotechnology technique.

However, the common well-plate ELISA has a limit of detection around single picomolar concentration. This limits the measuring capabilities of ELISA to the protein biomarkers that are abundant in samples. Additionally, since the reactivity of the enzymatic reporter is sensitive to trivial changes in the reaction conditions, a quantitative ELISA requires standard curves to be generated with each run, resulting in a limited sensitivity of ELISA to quantify small changes in the concentration of the analytes. Improvements in ELISA technology is needed to quantify protein biomarkers present in low concentrations, which could be indicative of early stages of diseases and its underlying immunological mechanisms.

Digital ELISA refers to a type of ELISA platform that allows the detection of measurement of protein biomarkers at a single-molecule level, by partitioning the reaction solution into a large number of picoliter to attoliter microreactors, so that most reactors are loaded with 0 or 1 analyte of interest dictated by Poisson statistics. Amplified signals are generated and accumulated within the compartments that contain at least one analyte so that by counting the number of signal-positive compartments against the signal-negative compartments, absolute quantification of the analyte of interest can be acquired.

The concept of enumeration of single protein molecules by discrete partitioning was first demonstrated by Rotman B. for β-D Galactosidase. See Rotman, B., Measurement of activity of single molecules of β-D-galactosidase, Proceedings of the National Academy of Sciences of the United States of America, 47(12), 1981 (1961). After microfluidic technologies enabled the reliable creation of microreactors in large quantities and with high uniformity, digital ELISA has been demonstrated by compartmentalizing samples using microwells, micro-valves and droplet emulsions. Various sizes of the microreactors have been created ranging from femtoliter to picoliter, and the detection limit has reached sub-attomolar.

However, the requirement of specialized equipment and techniques inhibited digital ELISA from being widely adopted. For example, the commercially available systems, such as SiMoA by Quanterix Corp. are often costly and limited in the variety of target biomarkers. For those who desire a higher level of in-house customization, extensive semiconductor fabrication skills and microfluidic techniques are needed to create microreactors, customize assay workflows to fit with the small volume, and to read signals from these microreactors. There is a need for a digital ELISA platform that's integrated with standard bench-top equipment and techniques, as well as widely available optical readout equipment, such as flow cytometers so that minimal training and new equipment is needed.

Particles have been integrated into immunoassay workflows, including digital ELISA workflows, usually only as a solid surface to capture analytes of interest and build immunocomplexes. For example, the SiMoA system by Quanterix Corp uses magnetic microparticles as a strategy to reduce the binding time for antigen-antibody reactions and to aid the loading of particles into microwells. Additionally, barcoding, via variations in colors or shapes were introduced to conduct particle-based multiplexed ELISAs. These methods are commercially sold by Luminex Corp as Luminex and Magplex technologies; and Abcam's FirePlex immunoassays. Particles have also helped with signal accumulation. For example, the Sysmex platform for droplet-free digital ELISA uses particles to capture reporter signals in addition to forming immunocomplexes. When the enzymatic reporter of the ELISA converts fluorescently-labeled tyramine to activated tyramide radicals, the radicals bind to tyrosine residues on the immunocomplexes, thus the fluorescent signals are immobilized on the particles. However, this approach lacks a compartmentalization strategy, causing crosstalk and loss of signal, and the formed radicals can be short-lived leading to loss of signal.

### SUMMARY

Here, a new platform or system is disclosed that uses hydrogel microparticles both as solid surfaces to build affinity complexes, and as hydrophilic cores to template the formation of water-in-oil emulsions (in some embodiments) and to capture signaling molecules within and/or on the microparticles. A complete workflow including the formation of affinity complexes, the generation and accumulation of amplified signals, and the signal readout can be performed using standard benchtop instruments and techniques. The democratized workflow also allows for customization towards a wider variety of targets of interest.

In one embodiment, a particle-based assay system for an analyte of interest includes a plurality of hydrogel microparticles having analyte capturing agents disposed on or within the hydrogel microparticles specific to the analyte of interest. The system further includes a catalytic reporter that forms an affinity complex with captured analyte of interest on or within the hydrogel microparticles. The system also includes substrate molecules that react with the catalytic reporter to generate one or more signaling molecules. The hydrogel microparticles may be contained in an emulsion (e.g., oil-based droplets) in some embodiments.

In another embodiment, a method of performing an assay with the particle-based assay system includes the operations of: a) incubating the plurality of hydrogel microparticles in a sample solution containing the analyte(s) of interest; b) incubating the plurality of hydrogel microparticles with the catalytic reporter to form an affinity complex; and c) exposing the hydrogel microparticles to the substrate molecules that react with the catalytic reporter to generate one or more signaling molecules. The plurality of hydrogel microparticles, in some embodiments, may be contained in droplets (e.g., surrounded by an oil-based fluid). These droplets may then optionally be broken to release the hydrogel microparticles. The hydrogel microparticles may be visually analyzed. The hydrogel microparticles may also be analyzed using a flow cytometer or fluorescence activated cell sorter (FACS). In some embodiments, the sample containing the analyte of interest is added to dried hydrogel microparticles to encourage the concentration of analytes around the exterior of the hydrogel microparticle.

### BRIEF DESCRIPTION OF THE DRAWINGS

FIG. 1 schematically illustrates the general workflow of how the hydrogel microparticles are used as part of an assay. This includes: (1) formation of the affinity complex (i.e., sandwich structure on the hydrogel microparticles); (2) optional compartmentalization (in an emulsion); (3) signal amplification in which a substrate is converted into a signaling molecule (i.e., reporter product); (4) signal accumulation; and (5) signal readout (e.g., using a FACS or flow cytometer device or microscope).

FIG. 2A illustrates how dried hydrogel microparticles may be used to improve analyte binding efficiency. The dried hydrogel microparticles swell after being added into an aqueous solution (with the analyte), taking up a majority of the fluid volume, driving the analyte to the hydrogel microparticle surfaces where they can bind to signal capture moieties.

FIGS. 2B and 2C show schematics of the before and after of hydrogel microparticle swelling in an aqueous solution containing streptavidin-FITC molecules. Small molecules such as water molecules are able to penetrate and are drawn in to swell the hydrogel matrix, while larger molecules above a molecular weight cutoff such as streptavidin-FITC are excluded from the volume taken up by the swollen hydrogel microparticles, and thus bind to the hydrogel microparticle surface at increased efficiency.

FIG. 2D illustrates a bright-field image of hydrogel microparticles dried on a glass slide, the top half of the hydrogel microparticles swollen from re-hydration.

FIG. 2E illustrates a fluorescent image of streptavidin-FITC bound to the outside of the hydrogel microparticles, leaving a ring-shaped fluorescent signal around the hydrogel microparticles.

FIGS. 2F and 2G show bright-field images of the dried hydrogel microparticles on glass slides before and after rehydration. Rehydrated particles swell and take up more fluid volume.

FIG. 2H shows imaging analysis indicating hydrogel microparticles going through an 86% increase in diameter from rehydration (n=446). Scale bars in the microscopic images represent 100 μm.

FIG. 3A schematically illustrates the process of creating a particle-templated emulsion. From left to right, the addition of hydrogel microparticles suspended in an aqueous solution, the addition of the oil phase onto the hydrogel microparticle suspension, and the final emulsion templated by the hydrogel microparticles. Also illustrated is an enlarged view of particle-templated droplets with single-particles due to the shearing process during vigorous fluidic agitation such as pipetting.

FIG. 3B is a bright-field image of a particle-templated emulsion created by vigorous pipetting.

FIG. 3C is a histogram showing the size distribution of the droplets templated by the hydrogel microparticles demonstrating high uniformity.

FIG. 4A schematically illustrates the capture of analytes of interest on the hydrogel microparticle.

FIG. 4B schematically illustrates the capture of analytes of interest on the hydrogel microparticle inside a droplet. Inside a particle-templated droplet, the enzymatic reporter labeling the affinity complex turns substrate molecules into signaling molecules.

FIG. 4C schematically illustrates signaling molecules accumulating inside the particle-templated droplet.

FIG. 4D schematically illustrates how, in some embodiments, the signaling molecules are captured by the signal capture moieties on the hydrogel matrix of the hydrogel microparticles, and thus stay immobilized on the hydrogel microparticles after the emulsion is broken and hydrogel microparticles are resuspended in an aqueous phase.

FIG. 5A schematically illustrates the capture of the analytes of interest on the hydrogel microparticle that uses a protease-based system (FIGS. 5A-5D).

FIG. 5B illustrates that inside a particle-templated droplet, the protease labeling the affinity complex cleaves peptides comprising the substrate molecules into smaller cleaved peptide signaling molecules.

FIG. 5C illustrates how the cleaved peptides accumulate inside the particle-templated droplet and are captured by the signal capture moieties on the hydrogel matrix.

FIG. 5D illustrates how the signaling molecules stay immobilized on the hydrogel microparticles after the emulsion is broken and hydrogel microparticles are resuspended in an aqueous phase.

FIG. 6A schematically illustrates the capture of the analytes of interest on the hydrogel microparticle that uses a nucleic acid-based system (FIGS. 6A-6D).

FIG. 6B illustrates that inside a particle-templated droplet, the nucleic acid strand labeling the affinity complex will be the template for nucleic acid amplification.

FIG. 6C illustrates how the process of nucleic acid amplification generates a complex nucleic acid structure that presents fluorescent signals either by intercalating dyes or inherent fluorescent labels on dNTPs.

FIG. 6D illustrates how the fluorescently labeled complex nucleic acid structure stays immobilized on the hydrogel microparticle after the emulsion is broken and hydrogel microparticles are resuspended in an aqueous phase.

FIGS. 7A-7B illustrates signal readout by a fluorescence microscope. FIG. 7A shows microscopic images of particle-templated droplets (bright field), Alexa-488 labeled hydrogel microparticles (FITC channel), and QuantaRed signals generated by HRP bound particles (TRITC channel), scale bar indicates 100 μm. FIG. 7B shows analysis results of the digitized signals from the TRITC channel for each particle-templated droplet.

FIGS. 8A-8D illustrate signal readout using an On-Chip Sort flow cytometer. FIG. 8A shows particle-templated droplets are initially gated using forward scatter and FL2 channel. FIG. 8B illustrates that combining forward scatter and side scatter, signals from particle-templated droplets are further selected. FIG. 8C shows that the droplets containing single particles are gated using the height and width of forward scatter. FIG. 8D shows that signals from the particle-templated droplets are visualized in the FL4 channel, with signals in the FL2 channel indicating the existence of microparticles. FIG. 8D shows signals from a negative control sample of particles without accompanying droplets.

FIGS. 9A-9D show signal readout using a BD FACS Canto II flow cytometer. FIG. 9A illustrates particles are initially gated by combining forward scatter and side scatter, signals from particles are further selected. FIG. 9B illustrates how single hydrogel microparticles are gated using the area and width of forward scatter. FIG. 9C illustrates the signals from the particles are visualized in the FITC channel. FIG. 9C shows signals from a positive control sample when the hydrogel microparticles are not labeled by APC-Cy7. FIG. 9D shows the dose response signal generated by labeling the hydrogel microparticles with HRP at increasing concentrations (top to bottom) followed by tyramide signal amplification, compiled in FlowJo software.

FIG. 10 illustrates a step emulsification device that is used for the production of hydrogel microparticles. An organic base is used to adjust the pH to effectuate gelation of the hydrogel microparticles. Also illustrated is an image of the pre-gelled hydrogel microparticles that are formed using the step emulsification device. A graph showing the monodisperse diameters of the hydrogel microparticles is also shown.

FIG. 11 illustrates a flowchart showing exemplary methods of using the particle-based assay system.

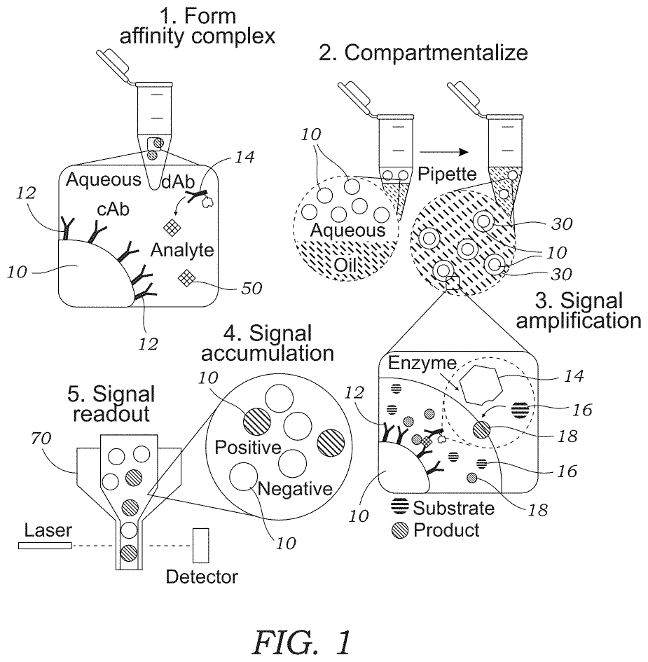

### DETAILED DESCRIPTION OF ILLUSTRATED EMBODIMENTS

The following definitions are used herein unless otherwise specified:

Reactive moiety(ies): functional groups modified onto the hydrogel matrix of the hydrogel microparticles that can react with and immobilize analyte capturing agents and/or signal capture moieties onto or within the hydrogel microparticles.

Signal capture moiety(ies): functional groups modified onto the hydrogel matrix of the hydrogel microparticles 10 that capture the signaling molecules 18 during signal accumulation.

Analyte capturing agent(s) 12: molecules exhibiting high affinity and specificity towards a matching analyte of interest 50, that when immobilized on the hydrogel microparticles 10 allow the hydrogel microparticles 10 to capture the analytes of interest 50 from the sample.

Sample(s): an aqueous solution containing the analytes of interest 50.

Analyte(s) of interest 50: the molecules inside the sample that is the target of detection, measurement, and/or analysis.

Linker molecule(s) 15: one or multiple molecules that attach catalytic reporter(s) to a captured analyte of interest via affinity binding.

Affinity complex(es): a complex of multiple molecules that may include one or multiple antigens, antibodies, aptamers, nucleic acids, etc. binding together as a result of affinity. An affinity complex including an analyte capturing agent 12, analyte of interest 50, and linker molecule 15.

Catalytic reporter(s) 14: When catalytic reporters 14 come in contact with substrate molecules 16, signal generation is initiated and signaling molecules 18 are produced. Is usually bound to the affinity complex to amplify signal.

Substrate molecule(s) 16: a type of molecule that reacts with the catalytic reporter 14.

Signaling molecule(s) 18: a type of molecule that is generated as a result of catalytic reporters 14 reacting with a substrate molecule 16.

Droplet(s): a dispersed phase formed by two immiscible phases, usually spherical in shape.

Particle-templated droplet(s) 30 or emulsion(s): an emulsion formed by dispersing an aqueous phase containing hydrogel microparticles in an oil phase, which causes substantially uniform droplets formed surrounding the hydrogel microparticles 10.

Satellite droplet(s) 32: background droplets generated during the emulsion formation process that do not contain any hydrogel microparticle 10.

In one embodiment, a particle-based assay system 2 is disclosed for one or more analytes 50 (i.e., an analyte of interest). The particles include a plurality of hydrogel microparticles 10 having analyte capturing agents 12 specific to the analyte of interest 50 disposed on or within the hydrogel microparticles 10. The hydrogel microparticles 10 have micrometer-sized diameters (in swollen or hydrated state). For example, in order to create the appropriate microcompartments for digital ELISA, the diameter of the hydrogel microparticles 10 is preferably smaller than 100 micrometers, more preferably in the range of 10-50 micrometers, more preferably between 20-30 micrometers. The hydrogel microparticles 10 are relatively homogenous in size, with a coefficient of variation (CV) of their diameters being smaller than 30%, preferably smaller than 15%, so that the difference in size does not increase the variations in signals. In some embodiments, especially when the hydrogel microparticles 10 have a wide size distribution, compensation can be performed using image processing software to normalize the signals against the microparticle size variation.

The hydrogel microparticles 10 produced are preferably spherical in shape so that the orientation of the hydrogel microparticle 10 does not affect signal readings, and substantially uniform spherical droplets can be formed around them. However, hydrogel microparticles 10 of alternative shapes can also be used, such hydrogel microparticles 10 with cavities, or shape-barcoded microparticles 10.

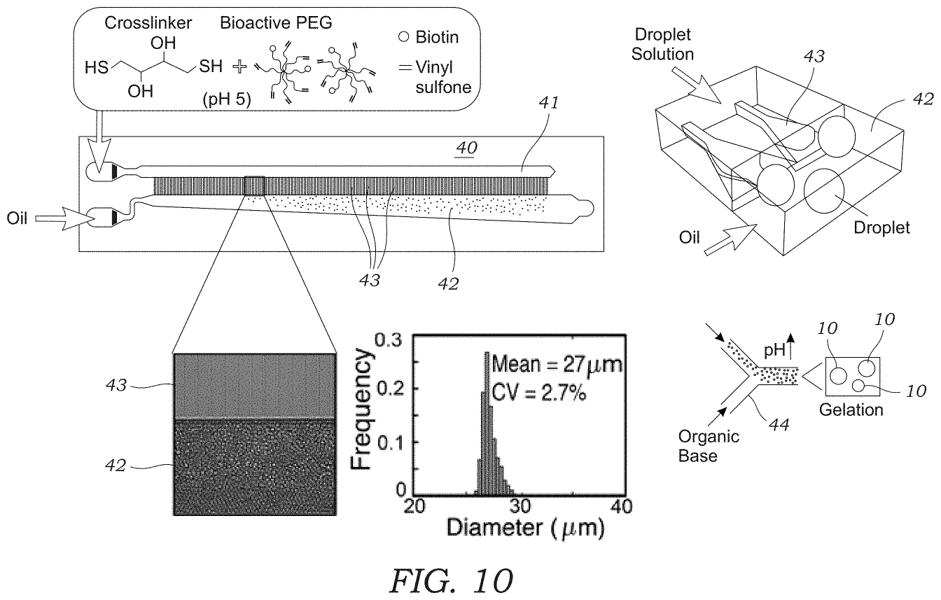

The hydrogel microparticles 10 are hydrogel-based, so that the hydrophilic properties of the hydrogel matrix can be leveraged to template a water-in-oil emulsion. The fabrication of the hydrogel microparticles 10 generally follows approaches outlined in International Patent Publication No. WO2020037214A1, which is incorporated herein by reference, for making monodisperse hydrogel microparticles. First, the hydrogel precursor materials needed for hydrogel microparticles 10 are dissolved into an aqueous solution in an un-crosslinked state. The aqueous solution is then emulsified in an oil phase to create an emulsion. After a stable emulsion is formed, a change of the environmental conditions is initiated to trigger the crosslinking of the hydrogel precursor materials, thus forming a hydrogel matrix in the shape of the volume of the aqueous droplets contained in the emulsion. The emulsion is then disrupted, and the gelled hydrogel microspheres that ultimately form the hydrogel microparticles 10 are washed to eliminate residual oil, surfactant, or unwanted aqueous or polymer components. The formation of an emulsion in the fabrication step can be carried out with common emulsion formation techniques known to those skilled in the art. In one embodiment, the emulsion of the aqueous polymer solution can be created using microfluidic emulsion devices, such as a step emulsification device 40 (FIG. 10) or a flow-focusing device. Previously, a parallelized step emulsification device enabled scalable high-throughput generation of monodisperse homogeneous spherical particles. See, e.g., International Patent Application No. PCT/US2021/029167, which is incorporated by reference herein. Flow focusing or droplet generating devices are disclosed in International Patent Publication No. WO2020037214A1, which is incorporated by reference herein. In another embodiment, the emulsion can also be made by sonication or vortexing and then hydrogel microparticles 10 are filtered to impose size constraints on collection of hydrogel microparticles 10.

The hydrogel materials used for the production of hydrogel microparticles 10 are important for forming a hydrophilic matrix for a particle-templated emulsion 30, and for providing a surface for the binding of affinity complexes. In addition, the hydrogel microparticles 10 should be hydrophilic in order to draw water into the matrix when starting in a dried state. Various polymers known in the art can be used to create hydrogel microparticles. These can include but are not limited to variations of poly(ethylene glycol) (PEG) polymers, variations of agarose, collagen, gelatin, alginate, variations of poly(acrylic acid) (PAA), etc.

The hydrogel microparticles 10 may be chemically functionalized with reactive moieties, such as biotin, streptavidin, carboxyl groups, etc., to introduce binding sites for the formation of affinity complexes on the hydrogel matrix of the hydrogel microparticles 10. These reactive moieties are covalently conjugated on some or all of the polymer chains that form that hydrogel matrix of the hydrogel microparticles 10 and exist in abundance. Biotinylation of the hydrogel microparticles 10 has been successfully demonstrated to allow for secondary streptavidin binding, by incorporating biotin-PEG-thiol within a solution of PEG-vinylsulfone and dithiothreitol (DTT) in a microfluidic droplet generation device. More specifically, the formation of biotin functionalized hydrogel microparticles 10 has been demonstrated by flowing an aqueous solution of 5 wt % 8-arm PEG-vinylsulfone, 2 wt % DTT, and 0.5 mg/mL 5 kDa biotin-PEG-thiol in a 0.15M triethanolamine (TEOA) buffer at pH 5, to be dispersed by an oil phase of 1% PicoSurf™ in NOVEC™ 7500 through a step emulsification device 40.

In some embodiments, the hydrogel microparticles 10 are chemically functionalized with signal capture moieties targeted at the signaling molecules 18 that are generated as a result of an amplified signal generation. These signal capture moieties are covalently conjugated on some or all of the polymer chains that form that hydrogel matrix of the hydrogel microparticles 10 and exist on and/or within the particles in abundance. These may be located within the pores of the hydrogel microparticles 10 and/or on the surface thereof. In some embodiments, tyrosine residues are used to capture tyramide radicals by incorporating a tyrosine containing peptide within a solution of PEG-vinylsulfone and dithiothreitol (DTT) in a microfluidic droplet generation device during hydrogel microparticle 10 formation. More specifically, the formation of biotin functionalized hydrogel microparticles has been demonstrated by flowing an aqueous solution of 5 wt % 8-arm PEG-vinylsulfone, 2 wt % DTT and 4 mM N-acetylated G-C-G-Y-G-R-G-D-S-P peptide [SEQ ID NO: 1] in a 0.15M triethanolamine (TEOA) buffer at pH 5, that was dispersed by an oil phase of 1% PicoSurf™ in NOVEC™ 7500 through a step emulsification device 40.

In some embodiments, the hydrogel microparticles 10 are also chemically functionalized with magnetic nanoparticles or microparticles contained therein, so that the hydrogel microparticles 10 or an emulsion templated by the hydrogel microparticles can be conveniently concentrated by placing a magnet (or magnetic field) in the vicinity of the hydrogel microparticles 10. These magnetic nanoparticles/microparticles can be either covalently conjugated on some or all of the polymer chains that form that hydrogel matrix of the hydrogel microparticles 10 or non-covalently trapped inside the gel matrix due to the limited porosity of the hydrogel.

In one embodiment, hydrogel microparticles 10 are stored suspended in a disperse phase, such as water, PBS buffer, or other aqueous solution. PEG-vinylsulfone hydrogel microparticles 10 may be stored in a PBS buffer containing 0.1% Pluronic-F127 to keep the hydrogel microparticles 10 from adhering to the wall of the Eppendorf or conical tubes used as containers. Hydrogel microparticles 10 stored using this method remain active for at least three (3) months.

In one embodiment lyophilization or drying can be used for long term storage of the hydrogel microparticles 10. This can enable control of hydrogel microparticle density in a given dispersed phase volume and allows for the dispersion of hydrogel microparticles 10 into an oil phase with high efficiency of encapsulation of the dispersed phase/sample fluid. Encapsulating hydrogel microparticles 10 in an emulsion prior to lyophilization avoids microparticle aggregation at resuspension. Specifically, Gelatin methacrylate droplets were microfluidically generated in NOVEC™ 7500 with 0.5% v/v PicoSurf™, leveraging the high vapor pressure and low freezing point of NOVEC™ 7500. The oil/surfactant-stabilized aqueous phase is deep-frozen (e.g., at −80° C. or −196° C.) and transferred to a lyophilizer to sublimate the ice and remove the volatile oil under vacuum (e.g., 0.06 mbar) for at least 6 h. This process results in a one-step conversation of emulsions to powders made up of micro-engineered hydrogel microparticles 10 with preserved properties.

In another embodiment, hydrogel microparticles 10 are dried in a solution of highly volatile solutions such as ethanol prior to being stored at −20° C. or −80° C. Crosslinked PEG-vinylsulfone hydrogel microparticles 10 were suspended in ethanol in a 1.5 ml Eppendorf tube, and then ethanol was dried out by blowing air at the opening of the tube using compressed air over 1-2 hrs. The hydrogel microparticles 10 are dried onto the wall of the tube. The tube is then closed and stored in a −20° C. or −80° C. freezer.

In order to introduce a sample with a new solution containing analytes 50 or any desired molecules to the hydrogel microparticles 10, the hydrogel microparticle 10 suspension is mixed with the new solution by pipetting, sometimes followed by vortexing, shaking or inverting to facilitate the even mixing of the two solutions. Optionally, hydrogel microparticles 10 can be concentrated prior to the addition of the new solution. The means of concentration include centrifuging the microparticle suspension and removing the supernatant, adding high-density components to the solution so that the hydrogel microparticles 10 can float to the surface and be collected, or magnetically fixing the hydrogel microparticles 10 in the vicinity of an external magnet if the hydrogel microparticles 10 are magnetically functionalized.

A method of size exclusion may be adopted to effectively increase the concentration of an analyte 50 from a sample, speed up the binding and enhance the binding efficiency (i.e., fraction of the analyte 50 that becomes bound) to the hydrogel microparticle 01. In one embodiment, the hydrogel microparticles 10 are lyophilized or dried. Sample containing the analyte of interest 50 can be introduced to dried hydrogel microparticle powder leading to re-hydration of the hydrogel microparticles 10. In another embodiment, the hydrogel microparticles 10 are first washed and suspended in ethanol. The ethanol content is then dried by blowing air, so that the hydrogel microparticles 10 dry on the surface of a substrate.

The hydrogel microparticles 10 shrink in size when they are dried. When the hydrogel microparticles 10 are rehydrated in an aqueous sample solution, sample fluid enters the hydrogel matrix, and the hydrogel microparticles 10 swell. However, in some embodiments, the hydrogel matrix pore size is tuned such that the analyte 50 is too large to enter into the interior of the hydrogel matrix. The hydrogel matrix pore size may be tuned or adjusted by controlling the cross-linking density of the hydrogel matrix that forms the hydrogel microparticles 10. Cross-linking density may be altered by controlling crosslinking conditions (e.g., crosslinking time, concentration of crosslinker, precursor molecular weight, etc.). The pore size of the hydrogel microparticles 10 is adjusted so that the pores have an average size or molecular weight cutoff or exclusion limit such that the analyte 50 is prevented from entering the interior of the hydrogel microparticles 10. In the context of pore size, the average pore size is smaller than an effective length (e.g., effective diameter) of the analyte 50. In the context of molecular weight, the pores should have a molecular weight cutoff or exclusion limit that is smaller than the molecular weight of the analyte 50. Note, however, that the pore size is such that, in some embodiments, it should allow the penetration of the signaling molecules 18 so that the signaling molecules 18 can be captured throughout the hydrogel microparticles 10.

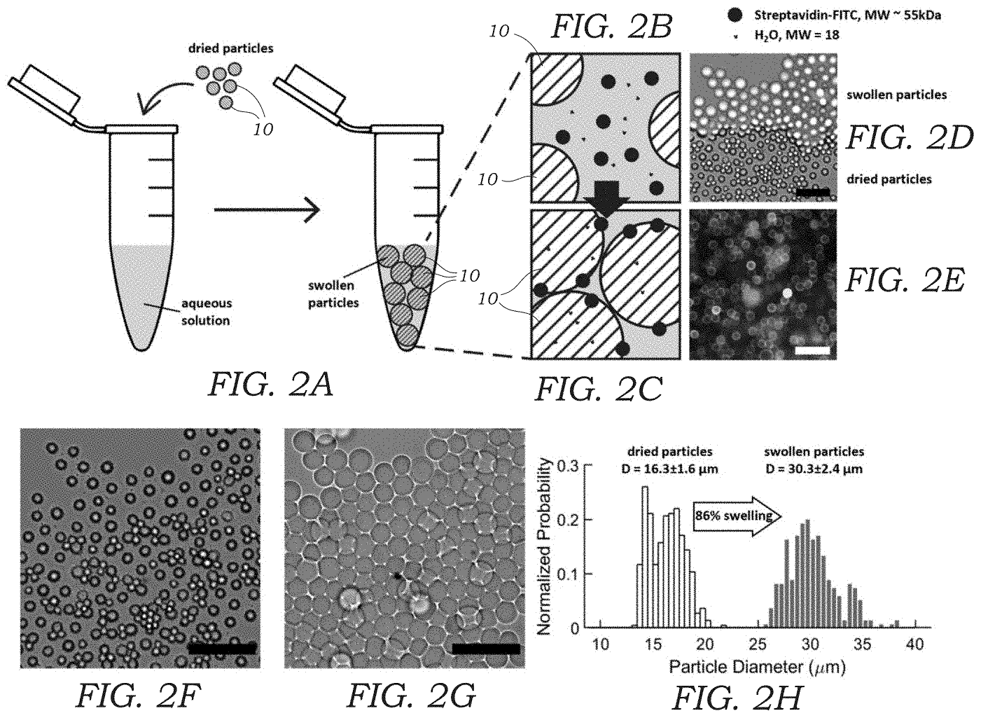

In the embodiment described above, the analyte 50 molecules are excluded from the space occupied by the swollen hydrogel microparticles 10, which leads to an increased concentration of analyte 50 molecules and an increased ability to bind to analyte capturing agents 12 on the surface of the hydrogel microparticles 10. This directed flow of sample fluid to swell the hydrogel matrix and size-based exclusion of target analytes 50 in the sample fluid can quickly drive them to hydrogel microparticle 10 surfaces where they can bind. In this embodiment, the sample fluid containing the analyte of interest 50 is added to the hydrogel microparticles 10 in a dried state to take advantage of the size-based exclusion of the analytes 50. The analyte of interest 50 is concentrated due to the fact that the aqueous solution that contains the analyte 50 enters the hydrogel microparticles 10 leaving less fluid volume outside the hydrogel microparticles 10 that contains the analyte 50 (e.g., FIGS. 2B and 2C).

For example, PEG-vinylsulfone hydrogel microparticles 10 suspended in ethanol have been dried, and rehydrated in a 1 mM solution of fluorescein-labeled streptavidin in PBS (FIGS. 2A-2H). The hydrogel microparticles 10 went through an 86% increase in diameter in the process of rehydration (FIGS. 2F-2G). Since 55 kDa streptavidin molecules are too big to penetrate the hydrogel matrix of the hydrogel microparticles 10, ring-shaped signals of FITC-streptavidin bound to the surface of the hydrogel microparticles 10 can be observed in the FITC channel after the hydrogel microparticles 10 are washed (FIG. 2E).

Washing steps are performed to remove unbound or non-specifically bound molecules such as analytes of interest 50, catalytic reporters 14, linker molecules 15, etc., thereby reducing background signals and enabling accurate measurements. To wash the hydrogel microparticles 10, the hydrogel microparticles 10 are first extracted from a solution containing undesired molecules. The means of extraction include centrifuging the microparticle suspension and removal of the supernatant, adding high-density components to the solution so that the hydrogel microparticles 10 can float to the surface and be collected, or magnetically fixing the hydrogel microparticles 10 in the vicinity of an external magnet/magnetic field if the hydrogel microparticles 10 are magnetically functionalized.

After the hydrogel microparticles 10 are extracted, a washing solution devoid of any interfering molecules (such as PBS buffer) is added to the hydrogel microparticles 10. The mixture is then agitated by pipetting, vortexing, reverting, sonication, or other fluidic agitation methods to enhance the mixing of hydrogel microparticles 10 with the washing solution. The hydrogel microparticles 10 are then extracted from the washing solution by the above-described extraction methods.

In some embodiments, the process of extraction—mixing with a washing solution—extraction can repeat several times, preferably greater than three (3) times, until the undesired molecules have been completely removed. Verification steps are optional to verify the complete removal of the undesired molecules from the solution surrounding the hydrogel microparticles 10. Examples of verification include colorimetric and fluorogenic chemical reactions, pH monitoring, sedimentation reactions, followed by optical or electrochemical measurements.

An incubation step usually follows a step of sample or reagent addition. The purpose of incubation is to provide enough time for the molecules in the solution to bind to the hydrogel microparticles 10, e.g., for the analyte capturing agents 12 to bind to the reactive moiety of the hydrogel microparticles 10, for the analyte of interest 50 in the sample to bind to the analyte capturing agents 12, for the catalytic reporter 14 to bind to the existing affinity complex, etc. In one embodiment, the hydrogel microparticles 10 mixed with the added solution are left at room temperature for an hour, to allow enough time for the desired molecules in the added solution to diffuse to the hydrogel microparticles 10 and be bound. Longer or shorter incubation times may be used. Generally, the incubation time for signal amplification is less than 24 hours. In some other embodiments, liquid agitation systems such as vortex mixers, shakers, rotators, etc. can be used, either sporadically or consistently throughout the incubation period, to ensure even distribution of analytes in solution during the incubation, and thus an even coating of the desired molecules onto the hydrogel microparticles 10. Such liquid agitation systems also enhance the mass transport and aid diffusion to enable better capture of analyte molecules.

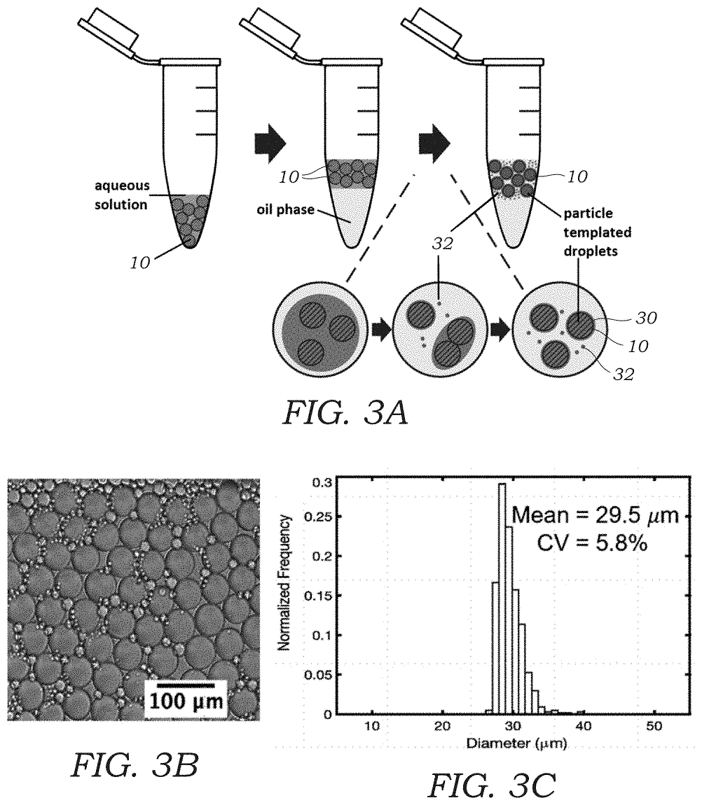

The formation of a particle-templated emulsion is achieved by combining a suspension of hydrogel microparticles 10 in an aqueous phase with oil (and optional surfactant) and mixing (e.g., by vortexing, pipetting, etc.) (FIG. 3A). Agitation and fluid dynamic shearing from mixing generates emulsions of decreasing size. After continued agitation, hydrogel microparticles 10 contained within the droplets 30 (i.e., particle-templated droplets 30) act as a size restraint that prevents further shrinking of the droplet 30. Particle-templated droplets 30 can also be thermodynamically stabilized. With increasing temperature or time, particle-templated droplets 30 do not coalesce in the same manner that unsupported drops of a dispersed phase in an aqueous phase will coalesce due to a decrease in interfacial energy of the system upon coalescence. Using mixing by pipetting and/or vortexing, uniform emulsions of particle-templated droplets 30 can be created along with smaller satellite droplets 32 containing no hydrogel microparticles 10. Due to their unique size range, particle-templated droplets 30 can easily be identified using image analysis or filtered from the surrounding smaller satellite drops 32. When the total aqueous volume of the sample is less than or equal to the volume that can be supported by the particle-templated droplets 30 mixed with the sample, satellite droplets 32 can be significantly reduced or eliminated.

The materials for the oil phase and surfactants can be chosen from oil and surfactants known to the field of emulsions. Preferably, fluorinated oil is used due to its low solubility to small molecules to prevent crosstalk among the emulsified droplets 30, and the surfactants matching the oil are preferably engineered for high stability. The concentration of the surfactants dissolved in oil needs to be optimized to stabilize the particle-templated emulsion, but not so excessive as to carry signaling molecules from one droplet 30 to another and cause signal crosstalk. In some embodiments, nanoparticles can be used to stabilize the emulsion interfaces and decrease the chance of crosstalk. The formation of particle-templated droplets 30 using various oil-surfactant systems has been demonstrated using the following: 7500 (3M company) oil with PicoSurf™ (Sphere Fluidics) surfactant at various concentrations from 0.1-2%, NOVEC™7500 with Fluo Surf (Emulseo) at various concentrations from 0.1-2%, and QX200 droplet generation oil (Bio-Rad Laboratories). All of these oil-surfactant systems enable stable particle-templated droplet 30 formation and render homogeneously sized droplets 30 (FIGS. 3B, 3C).

In some embodiments, it is preferred to remove the satellite droplets 32, the smaller droplets which are formed from aqueous phase sheared off of particle-templated droplets 30, using a variety of techniques. Due to the shearing process, satellite droplets 32 are often much smaller in size and therefore have a smaller volume, than particle-templated droplets 30. In one embodiment, the difference in size can be leveraged to separate satellite droplets 32 by filtration. A filter with a pore size that is too small to allow transport of particle-templated droplets 30 can be used to filter out satellite droplets 32 collected in the filtrate, while the desired particle-templated droplets 30 concentrate in the residue. In another embodiment, the difference in buoyancy can also be used to separate the satellite droplets 32 from particle-templated droplets 30. Since buoyancy scales with volume, the larger particle-templated droplets 30 float to the top of an emulsion quicker than smaller satellite droplets 30. After forming particle-templated droplets 30 (diameter of 29.5 μm), a 30-second incubation is enough to form a concentrated layer of desired particle-templated droplets 30 on the top of the emulsion. The bottom layer is then carefully pipetted out and discarded, and fresh NOVEC™7500+1% PicoSurf™ surfactant is added. This process significantly reduces, and in many cases eliminates, satellite droplets 32 from the emulsion. In another embodiment, magnetic forces can be leveraged to separate droplets 30 templated by magnetically functionalized hydrogel microparticles 10 from satellite droplets 32. In this case, hydrogel microparticles 30 are functionalized with magnetic nanoparticles in the manufacturing process. These are then used to template droplets 30 and emulsion is placed under an external magnetic field. Particle-templated droplets 30 are attracted to the magnetic poles while satellite droplets 32 remain afloat. The oil phase, containing satellite droplets 32, is then carefully pipetted and replaced with fresh oil.

In certain instances, it is desirable to return the hydrogel microparticles 10 back into a large volume of the dispersed phase (e.g., aqueous phase) in order to perform additional washing steps, secondary conjugations, run the hydrogel microparticles 10 through a flow cytometer 70 for analysis/sorting, etc. For a system 2 that includes particle-templated droplets 30, water, and fluorinated oil, a second surfactant such as perfluoro-octanol can be added into the oil phase to destabilize the particle-templated droplets 30 and collect the hydrogel microparticles 10 in the aqueous phase, and the suspension of hydrogel microparticles 10 can be directly removed from the top of immiscible phases that develop. If desired, additional washing steps can be performed with low-density organic phases miscible with the fluorinated oil, such as hexane and ethanol. Other methods such as centrifugation and destabilization via electric fields have been utilized to coalesce emulsions and could be potential alternative methods for the system. For example, the particle-templated droplets 32 may be broken by first mixing 20% perfluoro-octanol in NOVEC™ 7500 oil with the emulsion to destabilize the emulsion, followed by three rounds of washing using NOVEC™7500 and three rounds of extraction using hexane. Centrifugation is performed at the end of each washing step to concentrate the particles and to remove undesired liquid layers.

In the particle system 2 described herein, an affinity complex forms within or on the hydrogel microparticle 10. The affinity complex captures analyte(s) of interest 50 onto the hydrogel microparticles 10, and labels the captured analyte(s) of interest 50 with catalytic reporter(s) 14. An affinity complex refers to a complex formed from the binding of multiple agents, either covalently or noncovalently, including the analytes of interest 50. The structure of the affinity complex is controlled by selecting the agents with the desired affinity and specificity and binding the agents onto the particles in a designated order. The purposes of forming affinity complexes on the hydrogel microparticles 10 are (1) to capture the analyte(s) of interest 50, and (2) to introduce a catalytic reporter 14 to the hydrogel microparticles 10 that have captured one or multiple of the analytes of interest 50.

In order to capture the desired analyte(s) 50 from a sample on the hydrogel microparticles 10, the hydrogel microparticles 10 are first manufactured to comprise reactive moieties as described above and subsequently coated with analyte capturing agents 12. The analyte capturing agents 12 can be one or more of antigens, antibodies, aptamers, nucleic acids, or other molecules with affinity to the analytes 50. The hydrogel microparticles 10 with the analyte capturing agents 12 are then incubated with the sample so that the analytes 50 in the sample will bind to the analyte capturing agents 12. In one embodiment, the analyte capturing agents 12 are embedded into the hydrogel matrix of the hydrogel microparticles 10 during hydrogel microparticle fabrication. In this embodiment, the capturing agents 12 may be located through the interior and surface of the hydrogel microparticles 10. In another embodiment, the analyte capturing agents 12 are conjugated to the reactive moieties on the surface of the hydrogel microparticles 10 to form either covalent or non-covalent bonds. Examples of such binding reactions include EDC-NHS reactions, biotin-streptavidin binding, nucleic acid hybridization, azide-alkyne cycloaddition, etc. In this embodiment, the capturing agents 12 are conjugated to the hydrogel microparticles 10 after the hydrogel microparticles 10 have been formed.

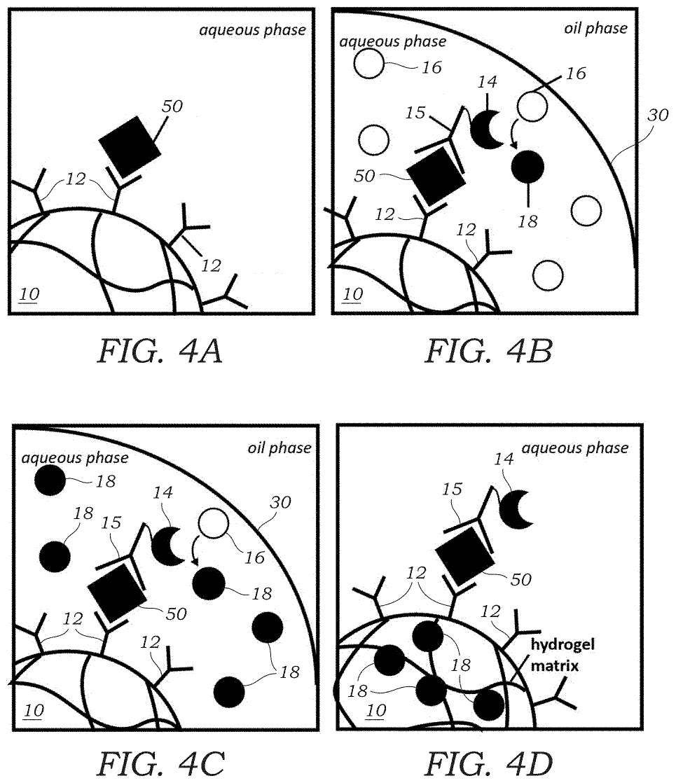

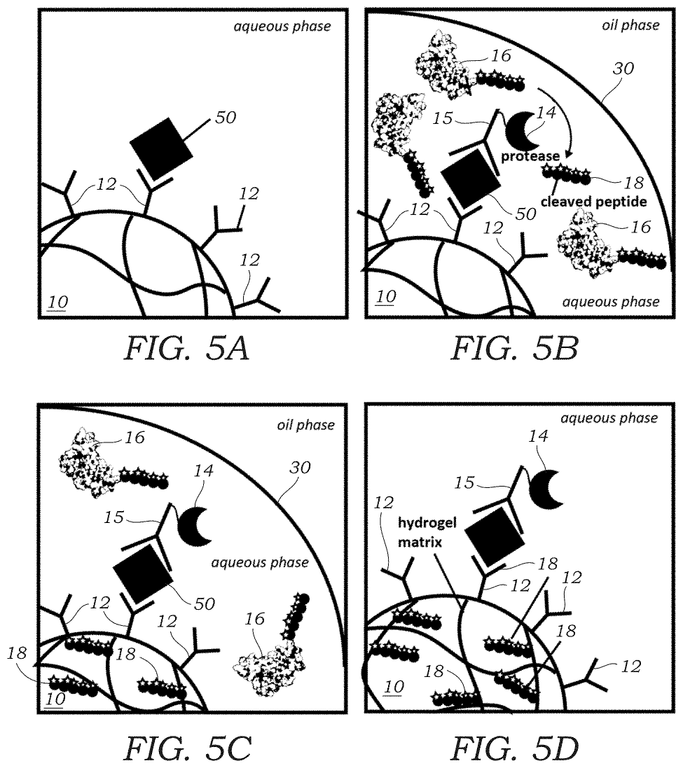

To introduce catalytic reporter molecules 14 to the affinity complex, one or more linker molecules 15 are used to link, i.e., label, the captured analytes 50 with the catalytic reporter molecules 14 (e.g., FIGS. 4A-4D and 5A-5D). The binding process can result in one or more catalytic reporter molecules 14 labeled on each affinity complex. In one embodiment, the introduction of the catalytic reporter molecules 14 includes introducing a single or a group of linker molecules 15 with an affinity to both the captured analyte of interest 50 and the catalytic reporter molecules 14. The linker molecules 15 can bind to the captured analyte 50 either covalently or noncovalently. The linker molecule 15 can either be pre-bound with one or multiple catalytic reporter molecules 14 or have a specific affinity to unmodified catalytic reporter molecules 14 that allows one or multiple catalytic reporter molecules 14 to bind. Examples of such linker molecules 15 include horseradish peroxidase (HRP)-labeled antibodies that are specific to the analyte of interest 50. In another embodiment, the introduction of catalytic reporter molecules 14 comprises introducing multiple linker molecules 15. The first linker molecule 15 binds with the analyte 50 captured on the hydrogel microparticles 10, the last linker molecule 15 binds to the previous linker molecule 15, and binds to catalytic reporter molecules 14 or is pre-labeled with one or multiple catalytic reporter molecules 14. Examples of such multi-linker structures include a mouse-sourced antibody specific to the analyte of interest, followed by a goat-anti-mouse IgG labeled with poly-HRP (a polymer comprising multiple horse radish peroxidase enzymes).

As described herein, a signal amplification process relies on substrate molecules 16 that react with the catalytic reporter molecule(s) 14 to generate one or more signaling molecules 18. The one or more signaling molecules 18 generate a visible signal in one embodiment. This may include the emission of fluorescence light in response to excitation light or other illumination. Signal amplification is a process wherein each catalytic reporter 14 bound to an immunocomplex generates large quantities of detectable signaling molecules 18 without the catalytic reporter 14 itself being consumed in the process. Catalytic reporters 14 act on substrate molecules 16 to generate signaling molecules 18. These signaling molecules 18 can be detected using standard laboratory equipment such as, but not limited to, fluorescence readers, microscopes 60, and flow cytometers 70. Signal accumulation is a strategy whereby the signaling molecules 18 are collected in a small volume or on hydrogel microparticle 10 surfaces or internal matrix to enable the concentration of signals from catalytic reporters 14 to detectable levels over the background. Signaling molecules 18 can be accumulated on or in the vicinity of hydrogel microparticles 10 provided there is a barrier to prevent loss of said signaling molecules 18.

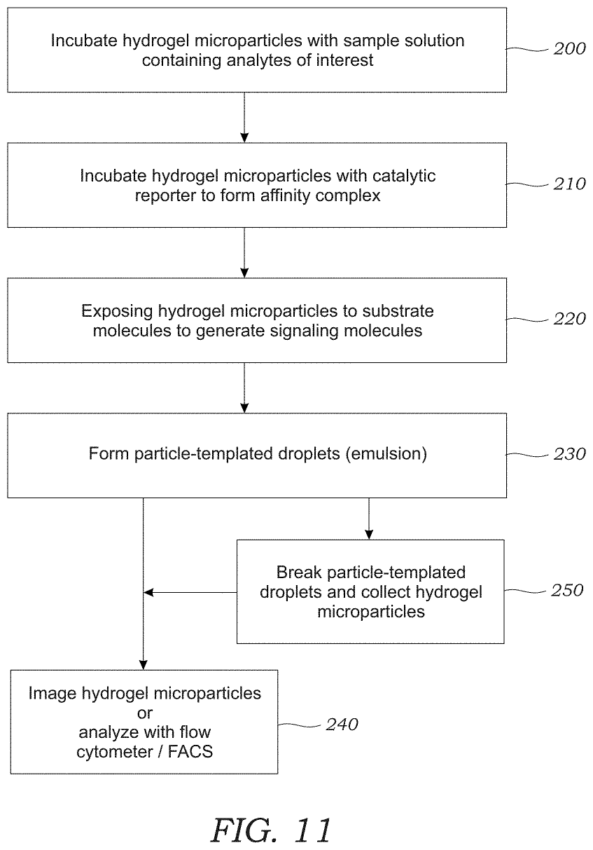

With reference to FIG. 11, a method of using a particle-based assay system 2 is illustrated. In operation 200, the hydrogel microparticles 10 are incubated with a sample solution that contains (or believed to contain) the analyte of interest 50. In one embodiment, the hydrogel microparticles 10 are initially in the dried state and hydrated with the sample. The hydrogel microparticles 10 may optionally be washed with, for example, a wash or buffer solution. The hydrogel microparticles 10 are then incubated with a catalytic reporter 14 to form an affinity complex as seen in operation 210. After this step, another optional wash is performed with a wash or buffer solution, the hydrogel microparticles 10 are exposed to substrate molecules 16 to generate signaling molecules 18 as seen in operation 220. Next, as seen in operation 230, an emulsion is formed with particle-templated droplets 30. The mixture of hydrogel microparticles 10 and substrate 16 may be pipetted vigorously (˜50 pipettes/minute) for 40 seconds to emulsify and form droplets 30. The particle-templated droplets 30 and the hydrogel microparticles 10 contained therein may then be imaged with an imaging device (e.g., microscope 60) or analyzed in a flow cytometer 70 (e.g., FACS) as illustrated in operation 240. A flow cytometer 70 generally includes a fluid conduit in which hydrogel microparticles 10 are continuously passed and analyzed using an incident laser or other light source. Optionally, prior to imaging and/or analysis in operation 240, the particle-templated droplets 30 are broken and the hydrogel microparticles 10 are collected for subsequent imaging and/or analysis as seen in this workflow path of FIG. 11.

### Enzyme Based System

When an enzyme-based system 2 is adopted for signal amplification, the catalytic reporter 14 can be selected from standard enzymatic reporters commonly used for ELISA, such as horseradish peroxidase (HRP), β-galactosidase (β-Gal), glucose oxidase, alkaline phosphatase (ALP), mutant or evolved versions of these enzymes, or a chemically modified version of these standard enzymatic reporters so that multiple enzyme molecules are linked, such as poly-HRP. The substrate molecules 16 are selected according to the selection of the catalytic reporter 14 so that the catalytic reporter 14 can convert the substrate molecules 16 into signaling molecules 18. For example, when HRP was selected to be the catalytic reporter 14, 10-acetyl-3,7-dihydroxyphenoxazine (ADHP) and its variations such QuantaRed™ (proprietary ADHP-based substrate 16) can be used as the substrate molecule 16, which can be converted by HRP to fluorescent resorufin as the signaling molecule 18; when β-Gal was selected to be the catalytic reporter 14, fluorescein-di-β-galactopyranosidase (FdG) or resorufin-β-galactopyranosidase (RβG) can be the substrate molecule 16, which can be converted by β-Gal into fluorescein or resorufin respectively as the signaling molecules 18. When ALP was selected to be the catalytic reporter 14, 4-methylumbelliferyl phosphate (4-MUP) can be used as the substrate molecule 16, which can be converted by ALP into 4-methylumbelliferone (4-MU) as the signaling molecule 18.

Following the formation of affinity complexes on the hydrogel microparticles 10, the hydrogel microparticles 10 are washed and resuspended in a signal development solution which contains the substrate molecules 16. Immediately following the addition of the signal development solution, the hydrogel microparticles 10 are optionally emulsified in an oil phase, and vigorously agitated to form an emulsion. If a hydrogel microparticle 10 has captured at least one analyte of interest 50, and therefore formed at least one affinity complex with one or multiple catalytic reporter 14 molecules, the catalytic reporter 14 will react with the substrate molecules 16 and generate a large number of signaling molecules 18 (FIG. 4B). If the hydrogel microparticles 10 are emulsified, the signaling molecules 18 can accumulate within the droplet 30 around the hydrogel microparticle 10 (FIG. 4C). In other cases, signaling molecules 18 accumulate in the vicinity of the hydrogel microparticle 10, but diffuse away from the hydrogel microparticle 10 into the surrounding solution unless otherwise captured by the hydrogel microparticle 10.

In one embodiment, signal accumulation is achieved by containing the signaling molecules 18 inside the emulsion droplets 30 (FIG. 4C). At the end of the signal generation, droplets 30 are imaged in a reservoir, container, or holder or using other analysis techniques described herein without breaking the emulsion. In one embodiment, signal accumulation is achieved by immobilizing the signaling molecules 18 onto the hydrogel matrix of the hydrogel microparticles 10. This can be achieved by including additional signal capture moieties on the hydrogel matrix of the hydrogel microparticles 10 that have selective affinity for the signaling molecules 18 over the substrate molecules 16 (FIG. 4D). For example, the hydrogel microparticles 10 further include anti-fluorescein antibodies on and/or within the hydrogel matrix. The anti-fluorescein antibody binds to fluorescein at the same epitopes that are initially blocked by the β-galactopyranose groups of FdG. The affinity of the anti-fluorescein antibody is higher to fluorescein than to FdG. In a related embodiment, anti-fluorescein antibodies are immobilized within the hydrogel matrix during fabrication and a fluorescein-conjugated to a sugar molecule with a molecular weight above a cutoff to enter the pores of the hydrogel matrix is used as the substrate molecule 16. Once fluorescein is released by β-galactopyranosidase, the molecular weight of the free fluorescein is sufficiently small to enter the pores of the hydrogel microparticle matrix and bind to the anti-fluorescein antibodies located within the hydrogel matrix. At the end of the signal generation and accumulation, in certain embodiments, the emulsion/droplet 30 can be disrupted, and the hydrogel microparticles 10 can be washed and imaged or analyzed as described herein in aqueous solutions.

### Protease Based System

When a protease-based system 2 is adopted for signal amplification, the catalytic reporter 14 can be a protease, and the substrate molecule 16 can be a fluorescently labeled peptide. The protease can cleave the peptide to expose an epitope to bind to a signal capture moiety such as an antibody or an aptamer. Following the formation of affinity complexes on the hydrogel microparticles 10 (FIG. 5A), the hydrogel microparticles 10 are washed and resuspended in a signal development solution that contains the substrate molecules 16, i.e., the peptides. Immediately following the addition of the signal development solution, the hydrogel microparticles 10 are optionally emulsified in an oil phase (e.g., to form particle-templated droplets 30), and vigorously agitated to form an emulsion. If a hydrogel microparticle 10 has captured at least one analyte of interest 50 and therefore formed at least one affinity complex with one or multiple catalytic reporter molecules 14, the catalytic reporter 14 will react on the substrate molecules 16 inside its surrounding droplet 30, and generate a large number of signaling molecules 18, i.e., activated peptides with the exposed binding epitope (FIG. 5B).

Signal accumulation is achieved by immobilizing the signaling molecules 18 onto and/or within the hydrogel matrix of the hydrogel microparticles 10 (FIG. 5C). The hydrogel microparticles 10 can include additional signal capture moieties on the hydrogel matrix that target the activated peptides so that the activated peptides can bind to the signal capture moieties on the hydrogel matrix. At the end of the signal generation and accumulation, the emulsion (i.e., droplets 30) can be disrupted, and hydrogel microparticles 10 can be washed and imaged or analyzed as described herein in aqueous solutions (FIG. 5D).

A variety of common affinity tags known to the field can be used in this system 2. For example, the catalytic reporter 14 can be an enterokinase, and the substrate molecule 16 can be a peptide or protein with a fluorescently labeled FLAG (peptide sequence DYKDDDDK [SEQ ID NO: 2]) tag. The substrate molecules 16 are of a high molecular weight that doesn't allow the substrate molecules 16 to penetrate the hydrogel matrix of the hydrogel microparticles 10. When an enterokinase 14 is presented on the affinity complex on the hydrogel microparticles 10, the enterokinase 14 can cleave off the fluorescently labeled FLAG tag which functions as the signaling molecule 18. The FLAG tag that is cleaved off is now of a small enough molecular weight to penetrate the hydrogel matrix of the hydrogel microparticle 10, and bind to the signal capture moieties i.e., anti-FLAG proteins such as M1, M2, and M5 or anti-FLAG antibodies.

### Kinase-Based System

When a kinase-based system 2 is adopted for signal amplification, the catalytic reporter 14 can be a kinase (e.g., ProQinase™ ABL1 (ProQinase GmBH, 0992-0000-1)) which phosphorylates a fluorescently labeled substrate molecule. This phosphorylated site can now be an epitope that can be recognized and bound to a signal capture moiety such as an antibody or an aptamer on the hydrogel matrix of the hydrogel microparticles 10. Following the formation of affinity complexes on the hydrogel microparticles 10, the hydrogel microparticles 10 are washed and resuspended in a signal development solution which contains the fluorophore conjugated substrate molecules 16 (e.g., ProQinase™ ATF2 (ProQinase GmBG,0594-000-2)), and ATP. Immediately following the addition of the signal development solution, the hydrogel microparticles 10 are optionally emulsified in an oil phase, and vigorously agitated to form an emulsion (i.e., particle-templated droplets 30). If a hydrogel microparticle 10 has captured at least one analyte of interest, and therefore formed at least one affinity complex with a catalytic reporter 14, the catalytic reporter 14 will react on the substrate molecules 16 inside its surrounding droplet 30, and generate a large number of signaling molecules 18, i.e., phosphorylated substrate molecules.

Signal accumulation is achieved by immobilizing the signaling molecules 18 onto the hydrogel microparticles 10. In one embodiment, the phosphorylated substrate molecules 18 can bind to the affinity complex structure on the hydrogel microparticles 10. In another embodiment, the hydrogel microparticles 10 can include additional signal capture moieties (e.g., Phosphotyrosine antibody, Genescript A01817) on the hydrogel matrix that target the phosphorylated substrate molecules 18, so that the phosphorylated substrate molecules 18 can bind to the signal capture moieties on the hydrogel matrix. At the end of the signal generation and accumulation, the emulsion can be disrupted, and hydrogel microparticles 10 can be washed and imaged or analyzed as described herein in aqueous solutions.

### Nucleic Acid-Based System

When a nucleic acid-based system 2 is adopted for signal amplification, the catalytic reporter 14 can be nucleic acid strands designed to template amplifications that result in complex nucleic acid structures, such as Loop-mediated isothermal amplification (LAMP), or rolling-circle amplification (RCA). The substrate molecule 16 is the individual deoxynucleoside triphosphates (dNTPs) which can form replicates of the DNA templates under appropriate assay conditions and can be bound to intercalating dyes to become signaling molecules 18. In some embodiments, the dNTPs can be itself fluorescently tagged, so that the fluorescence accumulates as the amplification proceeds.

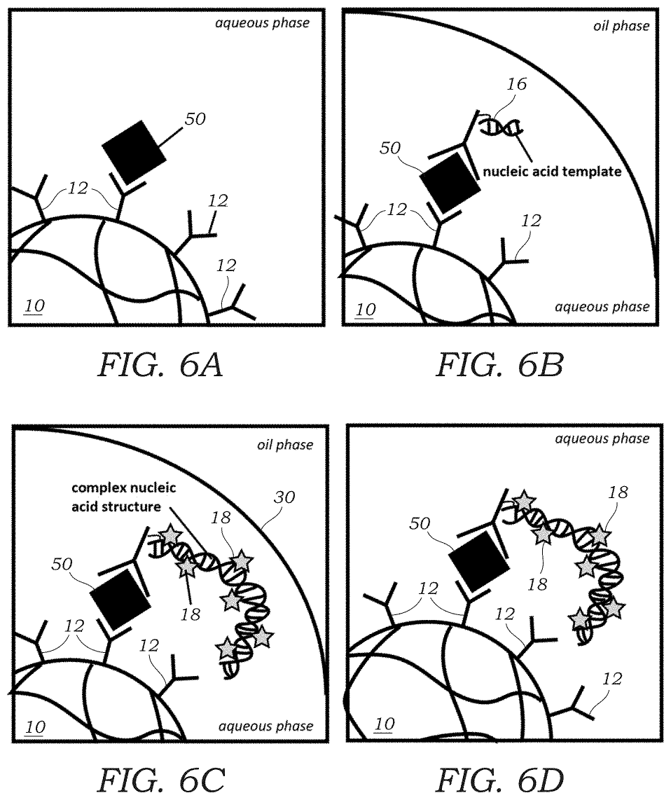

Following the formation of affinity complexes on the hydrogel microparticles 10 (FIG. 6A), the hydrogel microparticles 10 are washed and resuspended in a signal development solution that contains the substrate molecules 16, i.e., dNTPs and assay components necessary for LAMP or RCA. Immediately following the addition of the signal development solution, the hydrogel microparticles 10 are emulsified in an oil phase, and vigorously agitated to form an emulsion (i.e., particle-templated droplets 30) (FIG. 6B). The emulsion is kept at the proper temperature conditions for a designated period of time indicated by the protocol of LAMP or RCA. If a hydrogel microparticle 10 has captured at least one analyte of interest 50, and therefore formed at least one affinity complex with one or multiple catalytic reporters 14, a complex of complex nucleic acid structure will form from the amplification process, and provide signals either by the assembly of fluorescent dNTPs (which act as the signaling molecules 18) or by the integration of intercalating dyes 18 (FIG. 6C).

Signal accumulation is achieved by forming long DNA structures through a process of LAMP or RCA, which immobilizes the amplified DNA to the catalytic reporter 14, i.e., the original DNA template, which results in large amounts of fluorescent dNTPs or intercalating dye molecules 18 integrated into the complex DNA structure. At the end of the signal generation and accumulation, the emulsion can be disrupted, and hydrogel microparticles 10 can be washed and imaged or analyzed as described herein in aqueous solutions (FIG. 6D).

### Signal Readout and Sorting of the Particles

Signals generated and accumulated in the previously mentioned signal generation and accumulation steps can be detected using a multitude of either customized or commercially available readout methods. The readout is required to measure the output from the assay, and through calculation to provide information about the presence and/or concentration of the analytes 50 being detected from the sample.

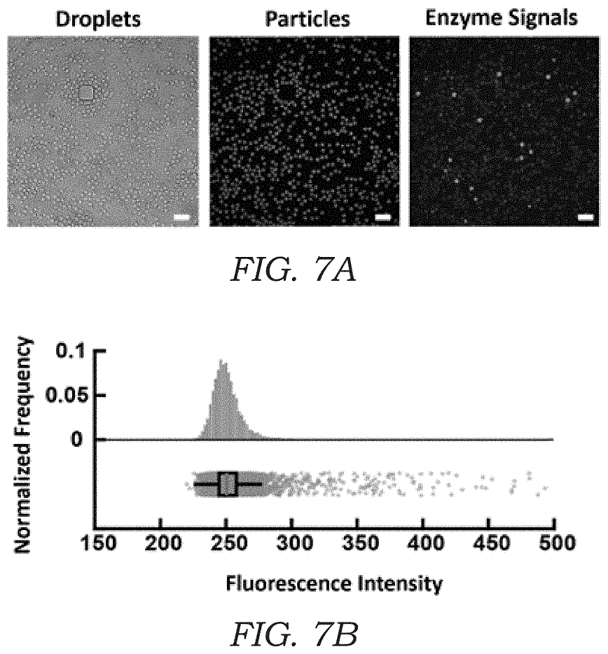

In one embodiment, fluorescence microscopes 60 or portable fluorescence readers can be used to obtain readouts of fluorescent signaling molecules 18. In such cases, droplets 30 can be pipetted onto a glass slide, in an imaging reservoir, on a cell-counting slide, or into any such containment unit which results in a single-layer distribution of the droplets 30, and then be imaged in the corresponding fluorescence channels to obtain signal readouts. This has been successfully demonstrated by pipetting emulsions onto glass slides, in PDMS reservoirs, and on cell-counting chips with a P200 pipette. In one embodiment, capillary forces can be used to fill a 50 μm deep glass capillary with emulsion. The containment units were placed onto the stage of a Nikon Ti-E fluorescence microscope 30 equipped with a Photometrics Prime sCMOS camera, and imaged in bright-field and fluorescent channels to capture images of droplets 30, fluorescently labeled hydrogel microparticles 10, and signals (FIG. 7A). Quantitative imaging analysis on signal intensity and distribution can be performed by Matlab (FIG. 7B).

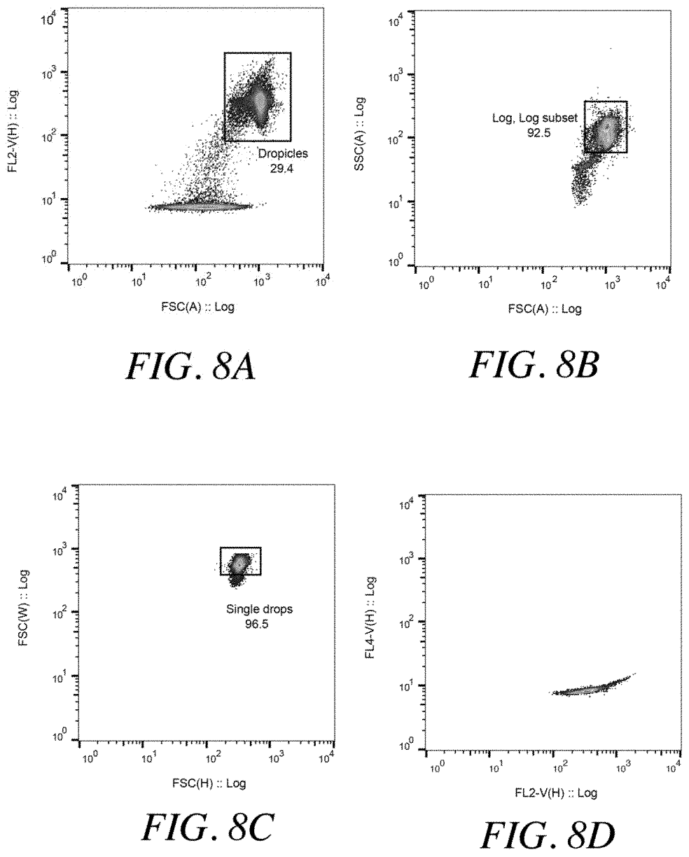

In another embodiment, high-throughput flow cytometers 70 and sorters that are compatible with the oils used as the continuous phase of the emulsion can be used to obtain readouts in such conditions where the assay involves emulsifying and emulsions are required to prevent leakage/loss of signal. Such flow cytometers 70 can have higher throughput and more sensitive sensors, such as photomultiplier tubes which enable faster and more sensitive fluorescent readouts (i.e., higher signal to noise ratio for the same samples). Faster data acquisition also quickens fluorescent readout and digital counting of a greater number of droplets, and thus enables more statistically accurate measurements at lower analyte concentrations where more particles need to be counted to ensure accuracy of measurement over the Poisson counting limit. In such a case, droplets 30 are flown through an oil compatible flow cytometer 70 and optical/fluorescence readouts are obtained. The use of OnChip Sort (OnChip Biotechnologies Co., Ltd.) has been successfully demonstrated to obtain fluorescent and scatter signal readouts from particle-templated droplets 30. Briefly, the emulsion was first loaded into a microfluidic chip (OnChip Biotechnologies, 80 μm (H)×80 μm (W)), and the chip was loaded into OnChip Sort according to the manufacturer's instructions. The readouts are first gated using forward scatter and FL2 (FIG. 8A), followed by a combination of forward and side scatters to distinguish particle-templated droplets 30 from smaller satellite droplets 32 and other noise (FIG. 8B), and then gated using the height and the width of forward scattering to distinguish singlets from doublets and/or triplets and bigger aggregates (FIG. 8C). Particle-templated droplets 30 can be further distinguished from satellite 32 droplets using a fluorescence dye linked to the hydrogel microparticles 10. Following gating on particle-containing droplets 30, gated signals from single particle-templated droplets 30 are evaluated in other fluorescence channels. Depending on the frequency of the fluorescent signals, the signals can be evaluated in its corresponding emission channels. Signal peak height and/or area is recorded, and droplets 30 with high signals give a proportional higher peak height and/or area (FIG. 8D). The number events of a fluorescence threshold value of peak height and/or area can then be counted, for example, to determine an analyte concentration. Alternatively, the intensity value for each event can be determined and the distribution or summary statistics of the distribution of events can be used to determine an analyte concentration.

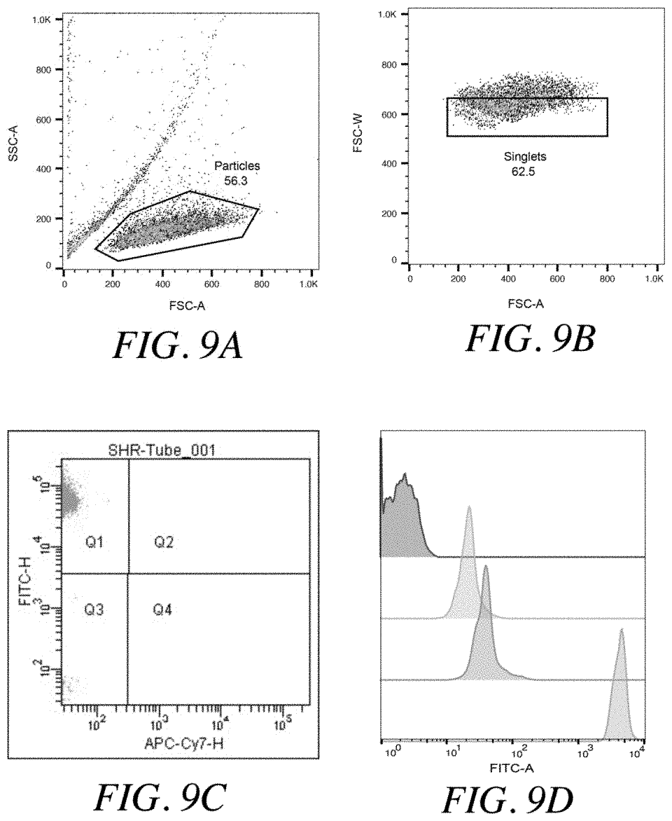

In another embodiment, readouts can be made compatible with standard flow cytometers 70 which only run aqueous solutions. In such cases, the signaling molecules 18 are captured onto the hydrogel microparticles 10 via the aforementioned signal capturing methods. Later, emulsions 30 are disrupted, and signals immobilized on hydrogel microparticles 10 can then be read out using standard flow cytometers 70. This readout method enables high-throughput readouts using standard flow cytometers 70 common in laboratories for single cell profiling. Dose-response readouts by varying concentrations of catalytic reporters 14 (horseradish peroxidase, and capturing fluorescent signaling molecules 18 (Alexa Fluor 488 labeled tyramide as activated radicals)) has been demonstrated on the surface of hydrogel microparticles 10 using tyramide chemistry. The signals were analyzed using a BD FACS Canto II cytometer at a throughput of 500 particles/second. The readouts are first gated using forward and side scatters to distinguish hydrogel microparticles 10 from dust, rare oil droplets and other noise (FIG. 9A), and then gated using the height and the width of forward scattering to distinguish singlets from doublets and/or triplets and bigger aggregates (FIG. 9B). Gated signals from single hydrogel microparticles 10 are evaluated in fluorescence channels. Depending on the frequency of the fluorescent signals, the signals can be evaluated in its corresponding emission channels (FIG. 9C). Signal peak height and/or area is recorded, and droplets with high signals give a proportional higher peak height and/or area. Fluorescence on hydrogel microparticles 10 is then outputted to be analyzed over a wide concentration range using e.g., FlowJo software (FIG. 9D). The number events of a fluorescence threshold value of peak height and/or area can then be counted for example to determine an analyte concentration. Alternatively, the intensity value for each event can be determined and the distribution or summary statistics of the distribution of events can be used to determine an analyte concentration.

### Barcoding Particles

Barcoding refers to creating different populations of hydrogel microparticles 10 with recognizably distinct traits, so that each population can be used to measure a type of analyte 50, and thus multiple analytes 50 and potential control reactions can be detected using multiple populations of hydrogel microparticles 10, enabling multiplexed signal detection. Hydrogel microparticles 10 can be barcoded in various ways.

In one embodiment, hydrogel microparticles 10 can be embedded with non-reactive and non-degradable nanoparticles of varying sizes or in varying concentrations. These can be read as differences in side scatter profiles by flow cytometers 70, or differences in imaging patterns using darkfield microscopy. Larger nanoparticles and/or higher concentration of nanoparticles produce higher side scatter.

In another embodiment, differences in the size of hydrogel microparticles 10 can be used to create size-based barcodes. These can be read as differences in forward scatter profiles on flow cytometers. On microscopes 60, these differences in sizes can be measured using image analysis software and coding scripts.

In another embodiment, varying concentrations of one or multiple fluorophores as signaling molecules 18 can be covalently conjugated to the hydrogel microparticle matrix. These produce different intensities of fluorescence in different fluorescent channels. These can be gated for and detected using image acquisition using fluorescence readers and image analysis software.

### Example: Detection of PSA by Digital ELISA on Hydrogel Microparticles

### 1. Fabrication of Microfluidic Step Emulsification Device

The step emulsification devices 40 were fabricated using soft lithography. The step emulsification device is formed by two parallel channels 41, 42 that are connected to one another via several hundred transverse channels 43 that form droplet nozzles where droplets are formed. A first channel 41 hold the pre-polymer and crosslinker. The second channel 42 holds an oil. The solution in the first channel 41 is flowed among hundreds of identical channels 43 (oriented transverse to long axis of channels 41, 42) and intersected by a taller reservoir channel 42 containing an oil, which enables the formation of monodisperse droplets at high rates. Master molds were fabricated on mechanical grade silicon wafers (University wafer) using a two-layer photolithography process with KMPR 1010 and 1050 (MicroChem Corp), the first and second layers defining the nozzle channel 43 height and the inlet/outlet reservoir region 41, 42 channel height, respectively. In the step emulsification device 40, the height of the outlet reservoir region 42 is higher than the nozzle channel 43 height. A nozzle channel 43 with length of 700 μm, width of 20 μm, and height of 7.2 μm was used. The droplet collection reservoir 42 measures 4 cm in length, 2 mm in width, and 80 μm in height using a Veeco Dektak 150 Surface Profiler. Devices 40 were molded from the masters using PDMS Sylgard 184 kit (Dow Coming). The base and crosslinker were mixed at a 10:1 mass ratio, poured over the mold, degassed, and cured at 65° C. overnight. The PDMS devices and glass microscope slides (VWR) were then activated via air plasma (Plasma Cleaner, Harrick Plasma) and bonded together. The bonded devices were then treated with Aquapel for 1 min and rinsed with NOVEC™7500 oil (3M) to render the channels fluorophilic. After modification, devices were placed in an oven at 70° C. for 1 h to evaporate residual oil in the channels.

For the imaging of droplets 30 and hydrogel microparticles 10, imaging reservoirs 5 cm in length, 3 cm in width and 50 μm in height were fabricated using the same technique to allow the droplets 30 or hydrogel microparticles 10 to form a single layer to be imaged.

### 2. Production of Hydrogel Microparticles

Hydrogel microparticles 10 were produced using hydrogel precursor solutions made from a 0.3 M TEOA (pH 5, Sigma-Aldrich) solution containing 10 wt % 8-arm PEG-vinylsulfone (PEG-VS, JenKeM Technologies). Dithiothreitol (DTT, Sigma-Aldrich) crosslinker was dissolved in deionized DI water at 4 wt %, calculated to occupy 80% of the vinyl sulfone groups, and pre-reacted with 10 μM Alexa Fluor 488 maleimide (Life Technologies) as a fluorescent label for the hydrogel microparticles 10. Precursor and crosslinker solutions were then added together at equal volume and mixed by vortexing. The combined solution was injected at 50 μL/hr along with a continuous phase composed of NOVEC™7500 oil (3M) and 0.5 wt % PicoSurf™ (Sphere Fluidics) at 100 μL/hr to generate water in oil emulsions using the step emulsification device 40 described herein.

The outlet tubing (polytetrafluoroethylene, Zeus) was connected to a Y junction 44 (FIG. 10) (IDEX Health & Science) where a second continuous phase containing 3% vol/vol triethylamine in NOVEC™7500 oil was introduced to increase the pH of the gel precursor droplets to pH 8.1 and initiate gelation. Oil containing the organic base was injected using a Hamilton gas-tight syringe at 25 μL/hr. The residence time in the tubing between introduction of the organic base and the collection tube was ≈4 min. All solutions were injected into the step emulsification device at defined flow rates using syringe pumps (Harvard Apparatus PHD 2000).

After incubation for 8 hr at room temperature, crosslinked hydrogel microparticles 10 were extracted from the oil using a series of washing steps. Excess oil was removed by pipetting and a solution of 20 wt % perfluorooctanol (Sigma-Aldrich) in NOVEC™7500 oil was added (approximately equal volume to remaining solution) to break down the emulsions 30. Phosphate-buffered saline (PBS, Thermo Fisher Scientific) was added to swell and disperse the hydrogel microparticles 10. The remaining NOVEC™7500 oil was removed by addition of hexane (Sigma-Aldrich) to lower the density of the oil, and hydrogel microparticles 10 were pelleted using a table top centrifuge at 2000×g for 5 min. Supernatant was removed and the hexane wash was repeated for 3 times. The fabricated hydrogel microparticles 10 were then filtered with Falcon™ 40 μm Cell Strainers (Corning) to remove oversized hydrogel microparticles 10, suspended in PBS supplemented with 1% Pluronic F-127 (Sigma Aldrich), and stored at 4° C. for long term storage up to several months.

For size characterization of the produced droplets 30 and hydrogel microparticles 10, droplets 30 or hydrogel microparticles 10 were collected in a second reservoir chamber and bright field images were taken using an inverted microscope (Nikon, Eclipse Ti-S fluorescence microscope).

### 3. Binding of Antibodies

Antibodies are purchased from Abcam (Anti-human, anti-PSA, monoclonal, Abcam: ab188388). Hydrogel microparticles 10 are first reacted with succinimidyl 3-(2-pyridyldithio)propionate (SPDP) (ThermoFisher Scientific, catalog no: 21857) (1 mL, 3.6 μg/mL) for 30 minutes. Later, particles are washed 4 times with PBS. Washed particles are reacted with capture antibodies (cAb) (125 ng/mL, 1 mL dissolved in PBS). These are covalently linked onto hydrogel microparticles 10 using SPDP linker. Hydrogel microparticles 10 are then washed again 4 times with PBS.

### 4. PSA Detection with Particle-Templated Digital ELISA

500,000 hydrogel microparticles 10 decorated with anti-PSA capture antibody (cAb) as the analyte capturing agent 12 are incubated with a sample solution containing PSA antigen 50 and incubated for 1 hour on a rotating rack set at 10 rotations per minute. Later, the hydrogel microparticles 10 are washed four (4) times with PBS supplemented with 1% Pluronic F-127. These are then incubated with a 10 pM solution of anti-PSA detection antibodies (anti-human, monoclonal, Abcam: ab188388) conjugated to horseradish peroxidase enzyme (i.e., the catalytic reporter 14) for 1 hour on a rotating rack. After detection antibody (dAb) incubation, the hydrogel microparticles 10 are washed four (4) times with PBS supplemented with 1% Pluronic F-127. 2 μL of QuantaRed™ working solution, prepared by mixing 10 parts of peroxide, 10 parts of enhancer, and 1 part of ADHP, all ingredients supplied by the QuantaRed™ Enhanced Chemifluorescent HRP Substrate Kit (ThermoFisher) (i.e., substrate molecule 16), is mixed with the pellet. 50 μL of NOVEC™7500 containing 1% PicoSurf™ is quickly added to the particle-substrate mixture and pipetted vigorously (˜50 pipettes/minute) for 40 seconds to emulsify and form droplets 30. After a 30 second wait period, the bottom layer of satellite droplets 32 is removed and replaced with fresh NOVEC™+PicoSurf™ mixture. Droplets 30 are incubated for 10 minutes at room temperature protected from light before fluorescent output from resorufin (signaling molecule 18) is read.

### 5. Signal Readout Using Fluorescent Microscope

Imaging reservoirs with the size of 5 cm (L)×3 cm (W)×50 μm (H) are fabricated using a PDMS stamping technique as described above. An inlet and an outlet both with 0.5 mm diameter are punched diagonally on two far sides of the reservoir. The reservoir is first filled with 1% PicoSurf™ in NOVEC™7500 oil using P200 pipette, followed by post-incubation droplets 30 transferred by pipetting. The particle-templated droplets 30 spread out into a single layer inside the reservoir since the height of the reservoir does not allow 2 hydrogel microparticles 10 stacked vertically. The reservoir containing the emulsion is then placed on the imaging stage of a Nikon Eclipse Ti2 Series microscope 60 equipped with a Photometrics Prime CMOS camera, and scanned for twelve (12) consecutive fields of views. For each field of view, one image is taken in the TRITC channel with 40 ms exposure to record the QuantaRed™ signals, followed by one image taken in the FITC channel with 40 ms exposure to locate the hydrogel microparticles 10. Standard image analysis algorithms are automated using MATLAB to analyze the fluorescent signals of each particle-templated droplet 30 by averaging TRITC signals over the hydrogel microparticle 10 area identified in the overlapping FITC channel. A positive signal is determined by thresholding three (3_ standard deviations above the mean of the background signal. Positive signals as a fraction of total positive and negative signals is used to determine PSA analyte concentration in the sample.

### 6. Signal Readout Using On-Chip Sort

Microfluidic chips with 80 μm size channels are purchased from On-Chip Biotechnologies Co., Ltd, and prepared according to the instructions. A sample of the particle-templated droplets 30 is pipetted from the top layer of the emulsion where the aqueous droplets are the densest, and transferred to the sample loading well on the microfluidic chip. The microfluidic chip is then placed inside the cartridge and inserted into the On-Chip Sort (On-Chip Biotechnologies Co., Ltd) flow cytometer 70 instrument according to the instructions. The droplet signals are gated first by forward scatter and FL2, followed by forward scatter against side scatter to eliminate satellite droplets 32, then by forward scatter height against forward scatter width to isolate singlets. The positive signals locate in the FL2(+) FL4(+) quadrant, whereas negative signals locate in the FL2(+) FL4(−) quadrant. Positive signals as a fraction of total positive and negative signals is used to determine PSA analyte concentration in the sample.

While embodiments of the present invention have been shown and described, various modifications may be made without departing from the scope of the present invention. The invention, therefore, should not be limited, except to the following claims, and their equivalents.

1. A particle-based assay system for an analyte of interest comprising:
a plurality of hydrogel microparticles having analyte capturing agents disposed on or within the hydrogel microparticles specific to the analyte of interest;
a catalytic reporter that forms an affinity complex with captured analyte of interest on or within the hydrogel microparticles; and
substrate molecules that react with the catalytic reporter to generate one or more signaling molecules.

2. The particle-based assay system of claim 1, wherein the hydrogel microparticles are porous and the analyte capturing agents are located within pores of the hydrogel microparticles.

3. The particle-based assay system of claim 1, wherein the catalytic reporter comprises one of: horseradish peroxidase (HRP), β-galactosidase (β-Gal), glucose oxidase, and alkaline phosphatase (ALP).

4. The particle-based assay system of claim 1, further comprising a signal capture moiety disposed on or within the hydrogel microparticles.

5. The particle-based assay system of claim 1, wherein the catalytic reporter comprises β-galactosidase (β-Gal), the substrate molecules comprise fluorescein-di-β-galactopyranosidase (FdG), and the one or more signaling molecules comprises fluorescein.

6. The particle-based assay system of claim 1, wherein the catalytic reporter comprises β-galactosidase (β-Gal), the substrate molecules comprise resorufin-β-galactopyranosidase (RβG), and the one or more signaling molecules comprises resorufin.

7. The particle-based assay system of claim 1, wherein the catalytic reporter comprises horseradish peroxidase (HRP), the substrate molecules comprise 10-acetyl-3,7-dihydroxyphenoxazine (ADHP) or an ADHP-based substrate, and the one or more signaling molecules comprises fluorescent resorufin.

8. The particle-based assay system of claim 1, wherein the catalytic reporter comprises alkaline phosphatase (ALP), the substrate molecules comprise 4-methylumbelliferyl phosphate (4-MUP), and the one or more signaling molecules comprises 4-methylumbelliferone (4-MU).

9. The particle-based assay system of claim 1, wherein the catalytic reporter comprises horseradish peroxidase (HRP), the substrate molecules comprise fluorescently labeled tyramide.

10. The particle-based assay system of claim 9, wherein the hydrogel microparticle is functionalized with tyrosine residues.

11. The particle-based assay system of claim 1, wherein the plurality of hydrogel microparticles are contained in an emulsion.

12. The particle-based assay system of claim 11, wherein the emulsion comprises droplets containing the plurality of hydrogel microparticles and wherein the droplets are substantially uniform in volume.

13. A method of performing an assay with the particle-based assay system of claim 1, comprising:
a) incubating the plurality of hydrogel microparticles in a sample solution containing the analyte(s) of interest;
b) incubating the plurality of hydrogel microparticles with the catalytic reporter to form an affinity complex;
c) exposing the hydrogel microparticles to the substrate molecules that react with the catalytic reporter to generate one or more signaling molecules.

14. The method of claim 13, further comprising analyzing the hydrogel microparticles of (c) using a flow cytometer or fluorescence activated cell sorter.

15. The method of claim 13, wherein the plurality of hydrogel microparticles are washed between operations a) and b), and/or b) and c).

16. The method of claim 13, further comprising:
d) forming an emulsion of the hydrogel microparticles.

17. The method of claim 16, further comprising:
e) breaking the emulsion;
f) subjecting the hydrogel microparticles to visualization or fluorescence analysis.

18. The method of claim 16, further comprising subjecting the hydrogel microparticles in the emulsion to visualization or fluorescence analysis.

19. The method of claim 13, wherein the plurality of hydrogel microparticles in a) are introduced to the sample solution in an initially dried state.


SEQUENCE LISTING (2 sequences)

SEQ ID NO: 1 - protein, 10 aa, Artificial Sequence
  tyrosine containing peptide
GCGYGRGDSP

SEQ ID NO: 2 - protein, 8 aa, Artificial Sequence
  fluorescently labelled peptide
DYKDDDDK